(19) United States
(12) Patent Application Publication
(10) Pub. No.: US 2017/0224224 A1
(43) Pub. Date: Aug. 10, 2017

(54) METHOD OF OBTAINING SYMMETRIC TEMPERATURE CHANGE

(71) Applicant: Chu-Yih Yu, Taipei (TW)

(72) Inventor: Chu-Yih Yu, Taipei (TW)

(21) Appl. No.: 15/015,136

(22) Filed: Feb. 4, 2016

(51) Int. Cl. A61B 5/01; G01K 13/00; A61B 5/00

(52) U.S. Cl. CPC A61B 5/01; A61B 5/688; A61B 5/742; A61B 5/0022; G01K 13/002; A61B 2562/0271

(57) ABSTRACT

A method of obtaining symmetric temperature change, comprising the following steps of preparing a temperature sensing device; measuring and collecting temperature change: placing the temperature sensing device (1) on a predetermined position of a participant to measure and collect the participant's body temperature change data; extracting data; removing the temperature sensing device from the participant's body when running out of the battery and then connecting a reading device to extract the body temperature change data out of the temperature sensing device in order to apply the body temperature change data in precision medicine; the body temperature change data is used as the key marker without affecting the participant's daily activity, preventing the participant from inaccuracy of the body temperature change data caused by psychological and/or physiological factors, so the body temperature change data can be applied in the symmetry precision medicine.

### FIELD OF THE INVENTION

The present invention relates to a method of obtaining temperature change, particularly to apply in precision medicine.

### BACKGROUND OF THE INVENTION

There is no two exactly the same green leaves, and there is no exactly the same gene between people, everyone taking the same medicine may not be effective, everyone's reactions to drugs are different, so in general, drug development from pharmaceutical companies or doctor's prescription, due to the technical limitations, can only treat the patient by “average level”, namely standardized medical care.

However, with the introduction of high-throughput sequencing technology and the development of large data processing capabilities, modern medical science is undergoing tremendous change from the traditional standardized medical care model to a personalized medicine paradigm, the personalized medicine that is “precision medicine” has become a new direction in modern medicine.

The purpose of the precision medicine is based on the patient's greatest wellbeing and the effective allocation of spending on socialized medicine, combined with modern epidemiology and preventive medicine, clinical diagnostics, therapeutics, molecular medicine, medical informatics technology, economics of health and medical sociology to let the traditional medicine move towards modernization and provide disease prevention, screening, diagnosis, treatment and rehabilitation program for everyone like tailored according to everyone's figure, with minimal resources to maximize health care, and improve the health of the overall population.

On the other hand, the Traditional Chinese Medicine (TCM) theory holds that the human body is viewed as a whole system that has nature operation and balance, you will get sick if your body is out of balance, and the “different treatment according to the same sickness nature” already exist in the therapeutic strategy of the TCM that is corresponding to the “precision medicine” theory, That's because everyone's physiques are different, the same treatment will generate different reactions and results, it is necessary for each patient to be diagnosed by pathology and etiology, in order to make the most appropriate treatment.

How can the precise medicine be combined with the TCM, combined the modern technology and traditional medical method with the scientific knowledge of the nature of the human body and disease to achieve the symmetry precision medicine, which is major problem.

In order to allow the TCM to achieve the precise medicine effect, it is very important to select the correct symmetric life information, which is a key marker of biology and physics; because the symmetric life information and the information gap [a state of balance] vary from person to person and from disease to disease, the medication and intervention are based on these situations, the so-called symmetric balanced medical care that has direction in personalized medicine.

So, to select symmetric temperature change as a key marker is a preferable choice, which has five following reasons, 1^{th}: humans are endotherms; 2^{th}: temperature is a statistics expression for the average kinetic energy of molecule; 3^{rd}: it is a greatest common divisor for a variety of medicine; 4^{th}: it is the most accurate, convenient, and cheapest way of life measuring way; 5^{th}: it is symmetry and symmetry breaking theory based on physics frontier in the 21st century.

Although it has been approved that the symmetric temperature change can help to realize the symmetry precision medicine. Currently, the biggest problem is there is no any method that can be used to obtain a symmetric temperature change.

In view of the above, how to effectively obtain a symmetric temperature change data and apply in the measurement of body temperature of the precision medicine has become the subject of the present invention intended to be improved.

### SUMMARY OF INVENTION

The present invention is to provide a method of obtaining temperature change with multi-point measurement at the same time for applying in the precision medicine.

For achieving the purpose of the present invention, the technical scheme discloses a method of obtaining symmetric temperature change, comprising the following steps of preparing a temperature sensing device, the temperature sensing device comprising a first sensing assembly, a second sensing assembly, and at a temperature sensing patch disposed on both first sensing assembly and the second sensing assembly;

measuring and collecting temperature change: placing the temperature sensing device on a predetermined position of a participant to measure and collect the participant's body temperature change data; the predetermined position comprising a first position group where the first sensing assembly is placed and a second position group where the second sensing assembly is placed; and extracting data: shutting down the power and removing the temperature sensing device from the participant's body; and then connecting a reading device to extract the body temperature change data out of the temperature sensing device, in order to apply the body temperature change data in precision medicine.

More particularly, wherein the certain period time (T) is at least 24 hours.

More particularly, wherein the first position group and the second position group symmetrically distribute on the participant's body surface.

More particularly, wherein the temperature sensing patch further comprises a temperature sensing terminal configured to attach to the human body surface for sensing temperature, a memory unit configured to store the body temperature change data, and a battery.

More particularly, wherein the temperature sensing patch comprises a housing, a circuit board, a processor chip connected with the circuit board, a lighting unit, and a port; wherein the housing comprises a receiving space disposed therein; wherein the circuit board is stalled in the receiving space and electrically connected with the temperature sensing terminal, memory unit, and battery; wherein the processor chip is installed on the circuit board and be capable of converting the body temperature change data from the continuing digital information of the temperature sensing terminal at a predetermined time and storing in the memory unit; wherein the light unit is installed within the receiving space and configured to indicate the operating status by lighting; and wherein the port is installed in the receiving space, one end of which is located at a periphery of the housing and configured to connect with the reading device.

More particularly, wherein the housing comprises a through hole corresponding to the temperature sensing terminal; wherein the temperature sensing patch further comprises an adhesive unit disposed on a surface of the housing and a periphery of the through hole, and configured to adhere to the human body surface; the adhesive unit comprising an adhesive layer and a release layer laminating with the adhesive layer.

More particularly, wherein the housing is divided into a first housing and a second housing correspondingly disposed on the first housing and having a receiving groove disposed in a side on top thereof; the receiving groove having an identification sticker configured to identify the temperature sensing patch.

More particularly, wherein the lighting unit is a LED light; wherein the port is one of the following: Type-A USB port, Type-B USB port, Type-C USB port, Micro-A USB, Micro-B USB, Mini-A USB port, Mini-B USB port.

More particularly, wherein the reading device comprises a case, a plurality of plug groups disposed in both sides on a top surface thereof, a screen disposed on the top side thereof, an output unit disposed within one side of the case, and a processing unit disposed within the case and electrically connected with the plug groups, screen, and output unit; wherein the plug groups comprises a plurality of plugs provided for the temperature sensing patch inserted therein to recharge the battery and the body temperature change data; wherein the processing unit is configured to process the body temperature change data and display on the screen, and/or transmit the data through the output unit.

Compared to prior art, the advantages of the present invention are the body temperature change data as the key marker without affecting the participant's daily activity, preventing the participant from inaccuracy of the body temperature change data caused by psychological and/or physiological factors, so the body temperature change data can be applied in the symmetry precision medicine; the design of the temperature sensing patch is small and practical that is attached to human body surface without affecting person's daily activity, moreover, the simple structure lowers the manufacturing cost that has greater valve of popularization and application.

### BRIEF DESCRIPTION OF THE DRAWINGS

FIG. 1 is a flow chart showing the steps of the invention;

FIG. 2 is an implementing schematic view of steps of preparing and measuring according to the invention;

FIG. 3 is an implementing schematic view of step of extracting according to the invention;

FIG. 4 is a perspective view of a temperature sensing patch according to the invention;

FIG. 5 is an exploded view of the temperature sensing patch according to the invention;

FIG. 6 is another perspective view of the temperature sensing patch according to the invention;

FIG. 7 is another exploded view of the temperature sensing patch according to the invention; and

FIG. 8 is a schematic view of a reading device according to the invention.

### DETAILED DESCRIPTION OF THE PREFERRED EMBODIMENTS

The following description is a preferable embodiment according to the Figs.

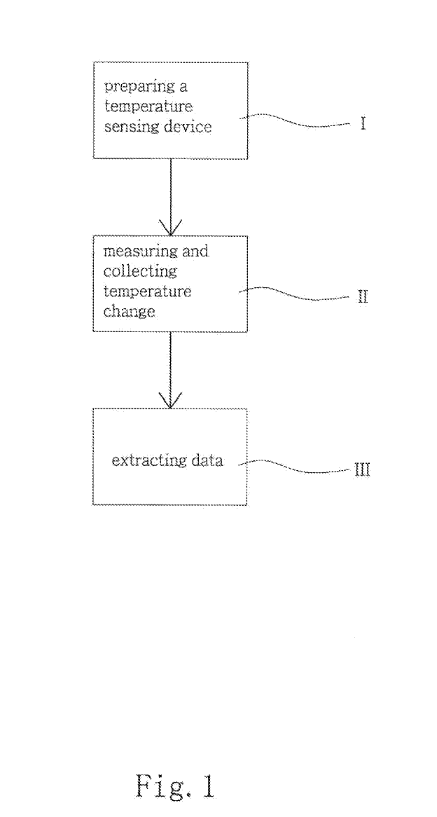

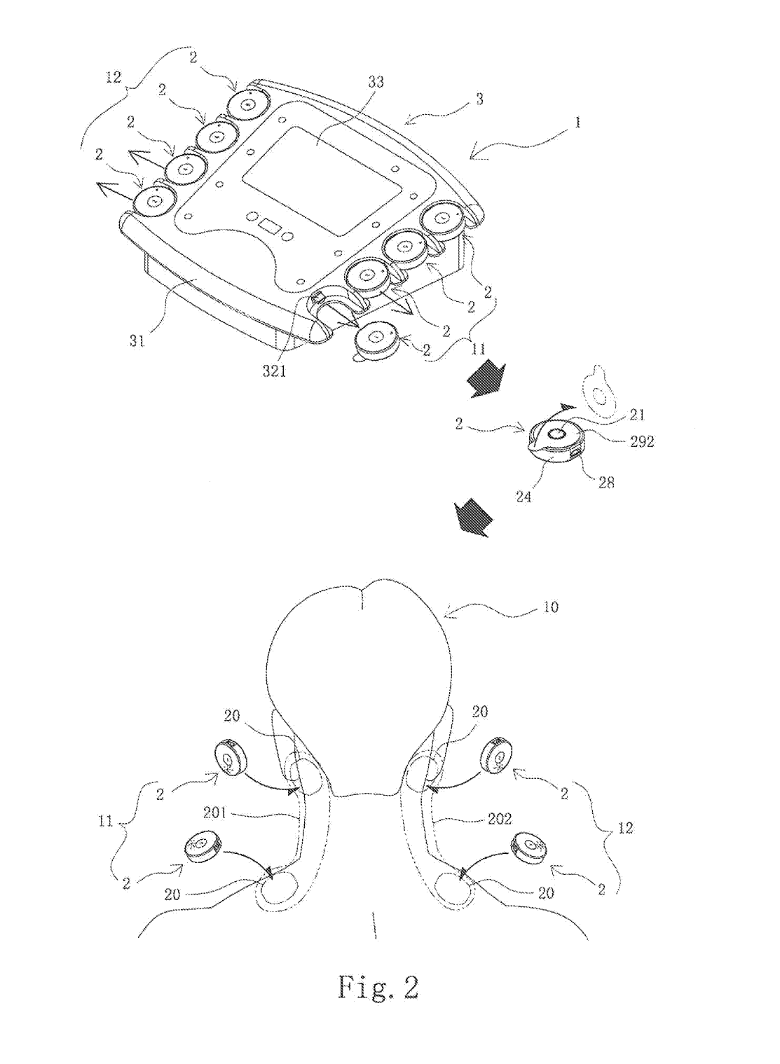

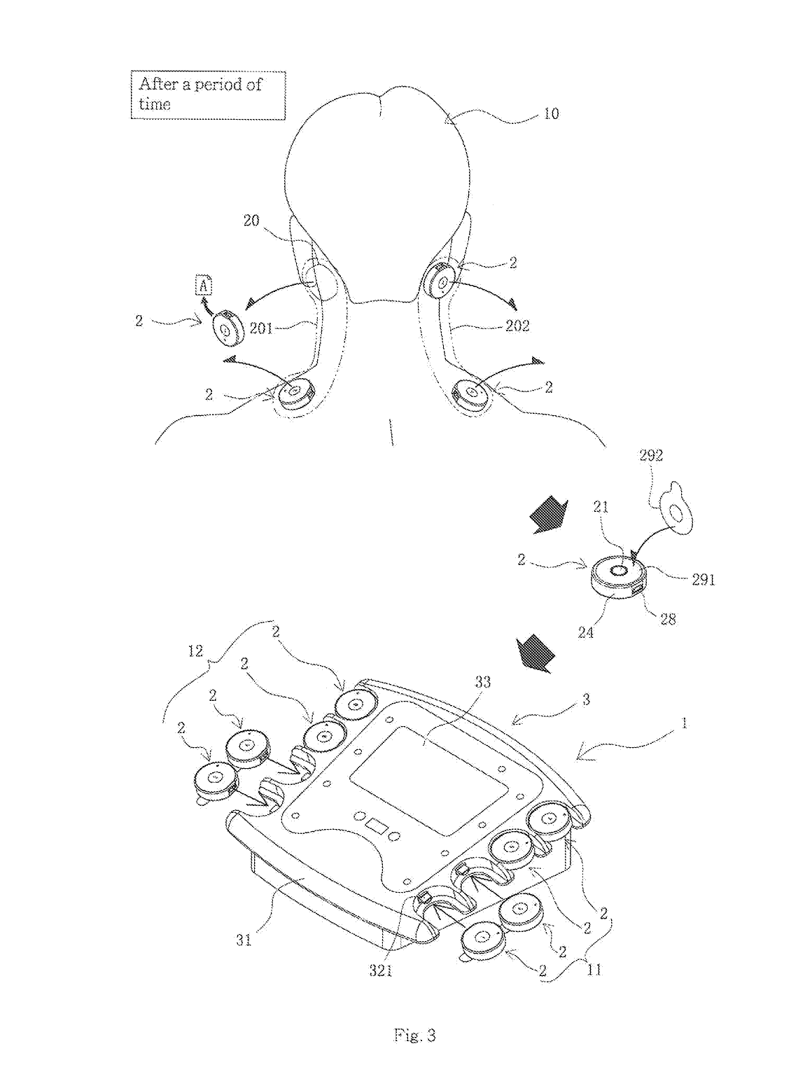

Referring to FIG. 1-3, the Figs. discloses a method of obtaining symmetric temperature change, comprising the following steps of preparing a temperature sensing device (1), the temperature sensing device (1) comprising a first sensing assembly (11), a second sensing assembly (12), and a plurality of temperature sensing patches (2) disposed on both first sensing assembly (11) and the second sensing assembly (12); measuring and collecting temperature change: placing the temperature sensing device (1) on a predetermined position (20) of a participant (10) to measure and collect the participant's body temperature change data (A); the predetermined position (20) comprising a first position group (201) where the first sensing assembly (11) is placed and a second position group (202) where the second sensing assembly (12) is placed; and extracting data: removing the temperature sensing device (1) from the participant's body after running out of the battery for a certain period of time (T), the temperature sensing device (1) has been stopped operation due to running out of power and the body temperature change data (A) has been collected; and then connecting a reading device (3) to extract the body temperature change data (A) out of the temperature sensing device (1), in order to apply the body temperature change data (A) in precision medicine.

The main objective of the steps is to obtain the body temperature change data (A) as the key marker without affecting the participant's (10) daily activity, preventing the participant (10) from inaccuracy of the body temperature change data (A) caused by psychological and/or physiological factors, so the body temperature change data (A) can be applied in the symmetry precision medicine.

The second point of the steps is no age limit, lower cost, and non-invasive measurement that can build a huge database in a short period of time, achieving the development of precision medicine so that everyone can enjoy this benefit.

The temperature sensing patch (2) do not set up manually power switch, therefore when running out of battery, the collection of the body temperature change data (A) is completed, thereby simplifying operation steps without always having to check the battery level.

The certain period of time (T) is at least 24 hours.

At least 24 hours of time as a reference to meet people's daily activity is beneficial in the body temperature change data (A), which can quickly determine the participant's physical condition and identify problems on the body.

Wherein the first position group (201) and the second position group (202) symmetrically distribute on the participant's body surface.

Because the human body itself has the characteristics of symmetry, it's quite obvious if the data is changed between the first position group (201) and the second position group (202), so as to facilitate determining the participant's (10) physical condition.

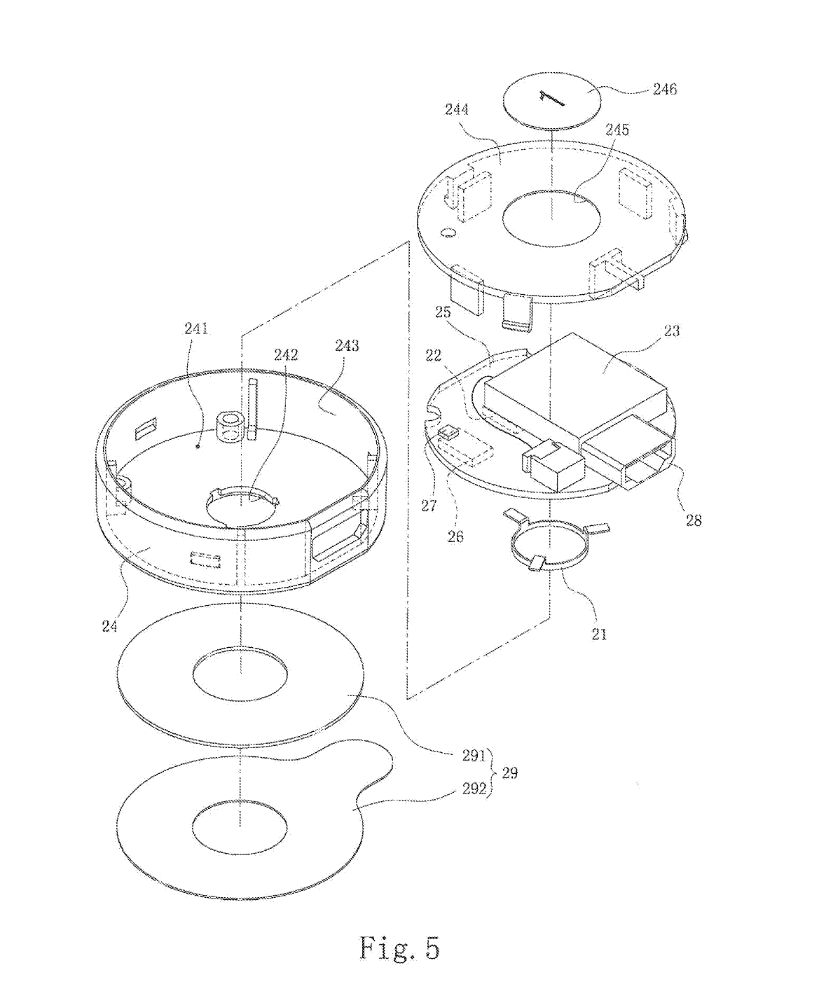

Referring to FIG. 5-7, wherein the temperature sensing patch (2) further comprises a temperature sensing terminal (21) configured to attach to the human body surface for sensing temperature, a memory unit (22) configured to store the body temperature change data (A), and a battery (23).

The temperature sensing patch (2) is used to measure the participant's (10) body temperature by the temperature sensing terminal (21), obtain the body temperature change data (A), and store the body temperature change data (A) through the memory unit (22); the entire operation process of the temperature sensing patch (2) is powered by the battery (23).

wherein the temperature sensing patch (2) comprises a housing (24), a circuit board (25), a processor chip (26) connected with the circuit board, a lighting unit (27), and a port (28); wherein the housing (24) comprises a receiving space (241) disposed therein; wherein the circuit board (25) is stalled in the receiving space (241) and electrically connected with the temperature sensing terminal (21), memory unit (22), and battery (23); wherein the processor chip (26) is installed on the circuit board (25) and be capable of converting the body temperature change data (A) from the continuing digital information of the temperature sensing terminal (21) at a predetermined time and storing in the memory unit (22); wherein the light unit (27) is installed within the receiving space (241) and configured to indicate the operating status by lighting; and wherein the port (28) installed in the receiving space (241), one end of which is located at a periphery of the housing (1) and configured to connect with the reading device (3).

Through the temperature sensing patch (2), the participant's body temperature change can be measured in a long period time to obtain the data for the precision medicine. Besides, the design of the small structure can reduce the burden and discomfort on the participant's body.

Unlike a thermometer of prior art, several temperature sensing patches (2) can be used simultaneously at a time to achieve the effect of multi-point temperature measurement, by which the temperature error generated by multi-point temperature measurement at different times will not happen.

Except for handling digital information transmitted from the temperature sensing terminal (21) within a predetermined time period, the processor chip (26) also can secure the data stored in the memory unit (22) before running out of the battery.

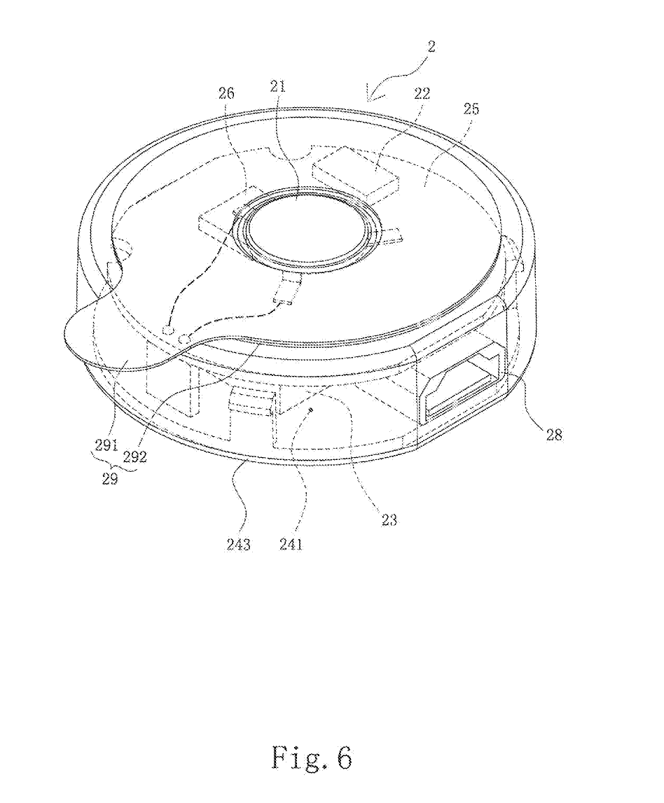

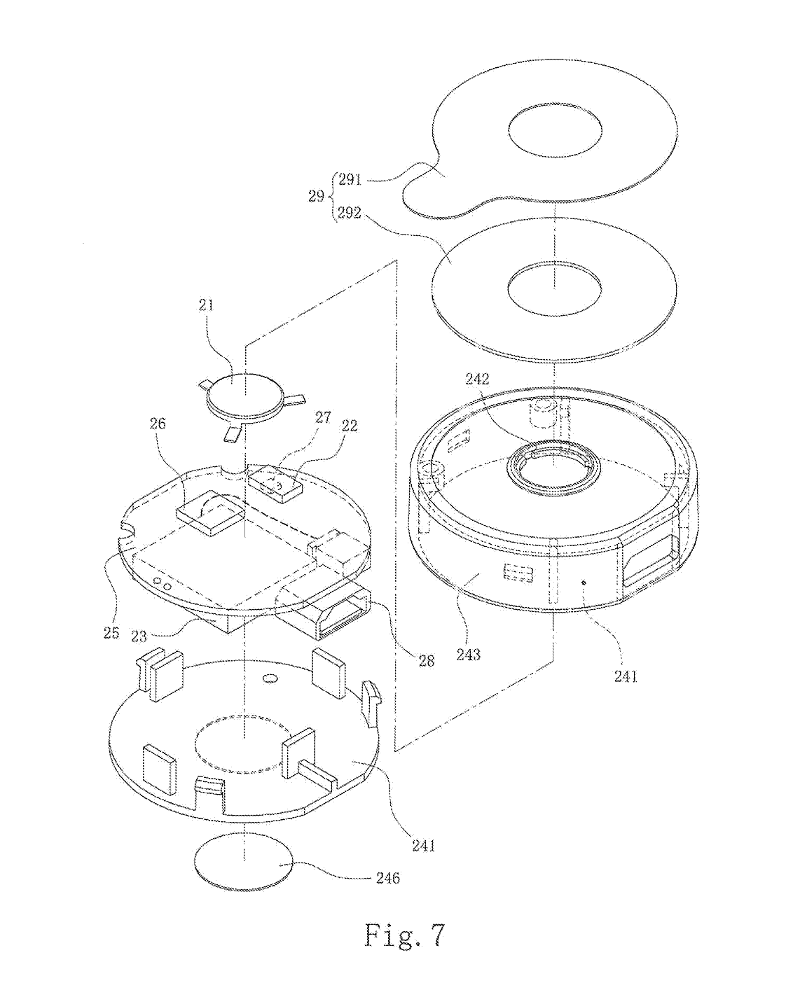

Referring to FIG. 6-7, wherein the housing (24) comprises a through hole (242) corresponding to the temperature sensing terminal (21); wherein the temperature sensing patch (2) further comprises an adhesive unit (29) disposed on a surface of the housing (24) and a periphery of the through hole (242), and configured to adhere to the human body surface; the adhesive unit (29) comprising an adhesive layer (291) and a release layer (292) laminating with the adhesive layer (291).

The through hole (242) let the temperature sensing terminal (21) directly contact with the participant's (10) skin, which can avoid the body temperature change data (A) error occurred. Besides, for complying with the medical standards, the adhesive unit (29) can be reusable or disposable.

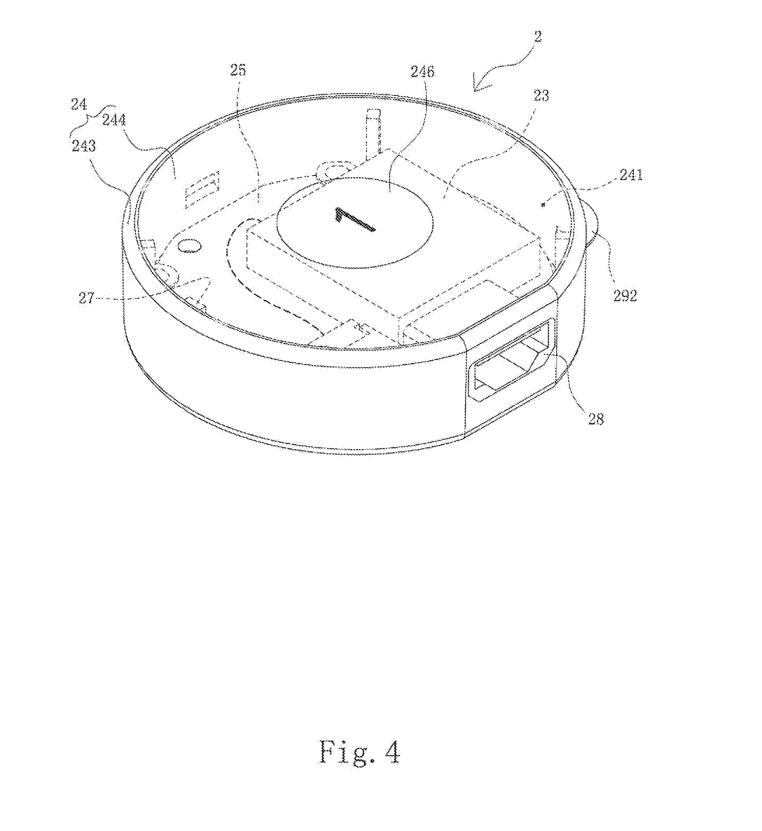

Referring to FIG. 4-5, wherein the housing (24) is divided into a first housing (243) and a second housing (244) correspondingly disposed on the first housing (243) and having a receiving groove (245) disposed in a side on top thereof; the receiving groove (245) having an identification sticker (246) configured to identify the temperature sensing patch (2).

The structure of the first and second housing (243, 244) is simple and easy to produce and assemble, and efficient to protect the internal devices; besides, in addition to making the temperature sensing patch (2) easy to fix, the receiving groove (245) can avoid the identification sticker (246) accidentally shedding off the second housing (244) caused by wear after using it for a long time.

Referring to FIG. 5-7, wherein the lighting unit (27) is a LED light; wherein the port (28) is one of the following: Type-A USB port, Type-B USB port, Type-C USB port, Micro-A USB, Micro-B USB, Mini-A USB port, Mini-B USB port.

The LED light applied in the light unit (27) not only can be easy to determine the operation status of the temperature sensing patch (2), but also can save more power and extend the use life. Besides, the USB applied in the port (28) not only can transmit the data and recharge the battery at the same time, but also can reduce the size of the temperature sensing patch (2).

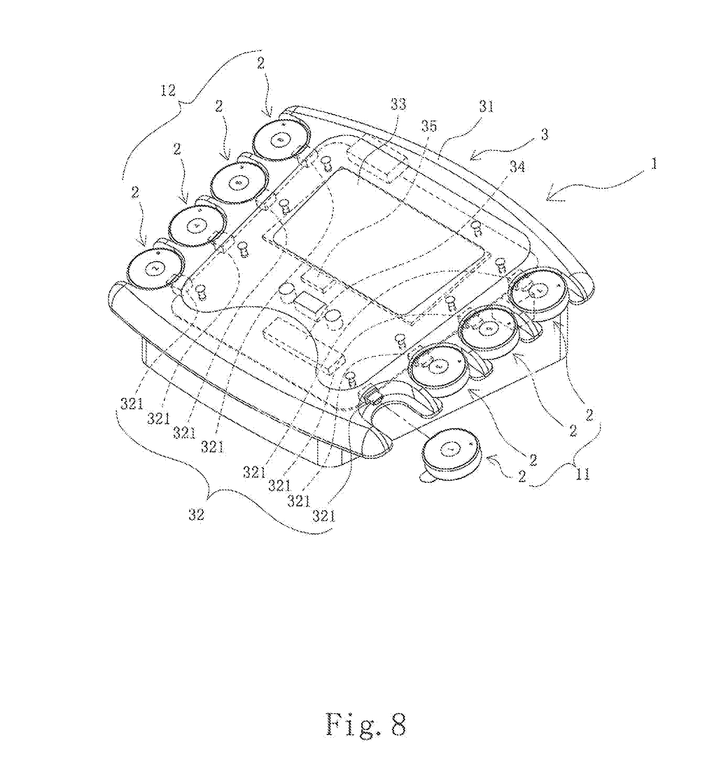

Referring to FIG. 8, wherein the reading device (3) comprises a case (31), a plurality of plug groups (32) disposed in both sides on a top surface thereof, a screen (33) disposed on the top side thereof, an output unit (34) disposed within one side of the case (31), and a processing unit (35) disposed within the case (31) and electrically connected with the plug groups (32), screen (33), and output unit (34); wherein the plug groups (32) comprises a plurality of plugs (321) provided for the temperature sensing patch (2) inserted therein to recharge the battery and the body temperature change data (A); wherein the processing unit (35) is configured to process the body temperature change data (A) and display on the screen (33), and/or transmit the data through the output unit (34).

It will be evident to those skilled in the art that the invention is not limited to the details of the foregoing illustrated embodiments and that the present invention may be embodied in other specific forms without departing from the spirit or essential attributes thereof. The present embodiments are therefore to be considered in all respects as illustrative and not restrictive, the scope of the invention being indicated by the appended claims rather than by the foregoing description, and all changes which come within the meaning and range of equivalency of the claims are therefore intended to be embraced therein.

I claim:

1. A method of obtaining symmetric temperature change, comprising the following steps of
preparing a temperature sensing device (1), the temperature sensing device (1) comprising a first sensing assembly (11), a second sensing assembly (12), and a plurality of temperature sensing patches (2) disposed on both first sensing assembly (11) and the second sensing assembly (12);
measuring and collecting temperature change: placing the temperature sensing device (1) on a predetermined position (20) of a participant (10) to measure and collect the participant's body temperature change data (A); the predetermined position (20) comprising a first position group (201) where the first sensing assembly (11) is placed and a second position group (202) where the second sensing assembly (12) is placed; and
extracting data: removing the temperature sensing device (1) from the participant's body after running out of the battery for a certain period of time (T), and then connecting a reading device (3) to extract the body temperature change data (A) out of the temperature sensing device (1), in order to apply the body temperature change data (A) in precision medicine.

2. The method of obtaining symmetric temperature change according to claim 1, wherein the certain period of time (T) is at least 24 hours.

3. The method of obtaining symmetric temperature change according to claim 1, wherein the first position group (201) and the second position group (202) symmetrically distribute on the participant's body surface.

4. The method of obtaining symmetric temperature change according to claim 1, wherein the temperature sensing patch (2) further comprises a temperature sensing terminal (21) configured to attach to the human body surface for sensing temperature, a memory unit (22) configured to store the body temperature change data (A), and a battery (23).

5. The method of obtaining symmetric temperature change according to claim 1, wherein the temperature sensing patch (2) comprises a housing (24), a circuit board (25), a processor chip (26) electrically connected with the circuit board, a lighting unit (27), and a port (28);
wherein the housing (24) comprises a receiving space (241) defined therein;
wherein the circuit board (25) is stalled in the receiving space (241) and electrically connected with the temperature sensing terminal (21), memory unit (22), and battery (23);
wherein the processor chip (26) is installed on the circuit board (25) and be capable of converting the body temperature change data (A) from the continuing digital information of the temperature sensing terminal (21) within a predetermined time and storing in the memory unit (22);
wherein the light unit (27) is installed within the receiving space (241) and configured to indicate the operating status by lighting; and
wherein the port (28) is installed in the receiving space (241), one end of which is located at a peripheral edge of the housing (1) and configured to connect with the reading device (3).

6. The method of obtaining symmetric temperature change according to claim 1, wherein the housing (24) comprises a through hole (242) corresponding to the temperature sensing terminal (21); wherein the temperature sensing patch (2) further comprises an adhesive unit (29) disposed on a surface of the housing (24) and a periphery of the through hole (242) for attaching on the human body surface; the adhesive unit (29) comprising an adhesive layer (291) and a release layer (292) laminating with the adhesive layer (291).

7. The method of obtaining symmetric temperature change according to claim 1, wherein the housing (24) comprises a first housing (243) and a second housing (244) correspondingly disposed on the first housing (243); the second housing (244) having a receiving groove (245) disposed in a side on top thereof; the receiving groove (245) having an identification sticker (246) configured to identify the temperature sensing patch (2).

8. The method of obtaining symmetric temperature change according to claim 1, wherein the lighting unit (27) is a LED light; wherein the port (28) is one of the following: Type-A USB port, Type-B USB port, Type-C USB port, Micro-A USB, Micro-B USB, Mini-A USB port, Mini-B USB port.

9. The method of obtaining symmetric temperature change according to claim 1, wherein the reading device (3) comprises a case (31), a plurality of plug groups (32) disposed in both sides on a top surface thereof, a screen (33) disposed on the top side thereof, an output unit (34) disposed within one side of the case (31), and a processing unit (35) disposed within the case (31) and electrically connected with the plug groups (32), screen (33), and output unit (34);
wherein the plug groups (32) comprises a plurality of plugs (321) provided for the temperature sensing patch (2) inserted therein to recharge the battery and the body temperature change data (A);
wherein the processing unit (35) is configured to process the body temperature change data (A) and display on the screen (33), and/or transmit the data through the output unit (34).
